SEQUENCE LISTING (11 sequences)

SEQ ID NO: 1 - RNA, 52 nt, Mus musculus
  source 1..52: mol_type = other RNA
gaaaggtggccggcaagagtcagtatcttctgggcggtatgacctgaccagt

SEQ ID NO: 2 - DNA, 25 bp, synthetic construct
  source 1..25: mol_type = other DNA
tggtcaggtcataccgcccagaaga

SEQ ID NO: 3 - RNA, 31 nt, Mus musculus
  source 1..31: mol_type = other RNA
gtggccggcaagagtcagtatcttctgggcg

SEQ ID NO: 4 - RNA, 540 nt, Homo sapiens
  source 1..540: mol_type = unassigned RNA
tgtctatacaataatgtttaggcatgccgatgttggaataacatggtacttaaaagacgatcagtaggattctgtctactttttaaaatcacataccctaaaaacattaacatttacagtaaatgtactgcttgactaaatatcagcattatttattaatctgggaaagagtgtcttggaaatagtttcagtgtgattaaactaaatttaaatggtttgtatctgaaaggtgaccggcaagagtcagtatcttctgggcggtatgacctaaccagttttacagctcatcaattactgctgcttgctttttgcataacaccccagaatgcttatcatttaagggagaataaaacagtacactgcaataatgtgttgtgtgttggctttgcctgtcactctttgaaacaagtttaaaataaacgctgcagttgcttttctattacattcactgcattaatgctacacataatgtcaacctaaatacaaagctataaagtcagttggataaataatggctagtacacattgtgagtggaggaa

SEQ ID NO: 5 - DNA, 23 bp, synthetic construct
  source 1..23: mol_type = other DNA
gagggcctatttcccatgattcc

SEQ ID NO: 6 - DNA, 20 bp, synthetic construct
  source 1..20: mol_type = other DNA
atgtttaggcatgccgatgt

SEQ ID NO: 7 - DNA, 22 bp, synthetic construct
  source 1..22: mol_type = other DNA
gctttgactttctgggaagatg

SEQ ID NO: 8 - RNA, 351 nt, Homo sapiens
  source 1..351: mol_type = unassigned RNA
acatttacagtaaatgtactgcttgactaaatatcagcattatttattaatctgggaaagagtgtcttggaaatagtttcagtgtgattaaactaaatttaaatggtttgtatctgaaaggtgaccggcaagagtcagtatcttctgggcggtatgacctaaccagttttacagctcatcaattactgctgcttgctttttgcataacaccccagaatgcttatcatttaagggagaataaaacagtacactgcaataatgtgttgtgtgttggctttgcctgtcactctttgaaacaagtttaaaataaacgctgcagttgcttttctattacattcactgcattaatgc

SEQ ID NO: 9 - RNA, 52 nt, Homo sapiens
  source 1..52: mol_type = unassigned RNA
gaaaggtgaccggcaagagtcagtatcttctgggcggtatgacctaaccagt

SEQ ID NO: 10 - DNA, 25 bp, synthetic construct
  source 1..25: mol_type = other DNA
tggttaggtcataccgcccagaaga

SEQ ID NO: 11 - RNA, 31 nt, Homo sapiens
  source 1..31: mol_type = unassigned RNA
gtgaccggcaagagtcagtatcttctgggcg

(19) United States
(12) Patent Application Publication
(10) Pub. No.: US 2026/0226471 A1
(43) Pub. Date: Aug. 6, 2026

(54) Compositions and Methods and Targeting a Splice Variant of the Regulatory Subunit of Myosin Phosphatase (MP) for Therapeutic Gain in Hypertension and Heart Failure

(71) Applicants: UNIVERSITY OF MARYLAND, BALTIMORE, Baltimore, MD (US); The United States Government as represented by the Department of Veterans Affairs, Washington, DC (US)

(72) Inventor: Steven Andrew Fisher, Columbia, MD (US)

(21) Appl. No.: 19/123,563

(22) PCT Filed: Oct. 27, 2023
(86) PCT No.: PCT/US2023/078072

§ 371 Date: Apr. 23, 2025

Related U.S. Application Data
(60) Provisional application No. 63/419,901, filed on Oct. 27, 2022.

(51) Int. Cl. C12N 15/113; A61K 31/712; A61K 31/7125; A61K 45/06; A61P 9/04; A61P 9/12

(52) U.S. Cl. CPC C12N 15/1137; A61K 31/712; A61K 31/7125; A61K 45/06; A61P 9/04; A61P 9/12; C12N 2310/11; C12N 2310/314; C12N 2310/315; C12N 2310/321; C12N 2310/3233; C12N 2310/346; C12N 2310/351; C12N 2320/31; C12N 2320/33

(57) ABSTRACT

The present invention provides an oligomer capable of binding to a target site of precursor messenger RNA (pre-mRNA) of myosin phosphatase target subunit (Mypt1) in cells, wherein binding of the oligomer to the target site of the pre-mRNA suppresses splicing of exon 24 of myosin phosphatase target subunit (Mypt1) in cells. Pharmaceutical compositions comprising the same and methods of use thereof are further provided.

### CROSS-REFERENCE TO RELATED APPLICATIONS

This application is a national phase filing under 35 U.S. C. § 371 of International Application No. PCT/US2023/078072, filed Oct. 27, 2023, which claims the benefit of U.S. Provisional Appl. No. 63/419,901, filed on Oct. 27, 2022, the contents of which are hereby incorporated by reference in their entirety.

### STATEMENT OF FEDERALLY SPONSORED RESEARCH AND DEVELOPMENT

This invention was made with government support under the Grant Number HL142971 awarded by the National Institutes of Health and a Merit Award BX004443 awarded by the United States Department of Veterans Affairs. The government has certain rights in the invention.

### REFERENCE TO SEQUENCE LISTING

The Sequence Listing submitted Apr. 16, 2026 as a text file named “37759.0434U2 updated SL.xml,” created on Apr. 15, 2026, and having a size of 11,821 bytes is hereby incorporated by reference pursuant to 37 C.F.R. § 1.52 (e) (5).

### FIELD OF THE INVENTION

The field of the invention relates to medicine and therapeutics, in particular to treatments for hypertension and heart failure.

### BACKGROUND OF THE INVENTION

Alternative exon usage (AEU) is present in most genes (Merkin, J., et al., Science, (2012), 338:593-9; Wright et al., Nature Reviews Genetics, (2022), 23:697-710) and a major driver of cellular, including smooth muscle cell, phenotypic diversity (Fisher, S. A, Adv Pharmacol, (2017), 78:383-415; Fisher, S. A., Physiol Genomics, (2010), 42A: 169-87). The thousands of alternative exons provide an attractive target for gene therapies that by suppression or activation of the alternative exon could shift the expression of naturally occurring protein isoforms for therapeutic gain. This approach is in use in the treatment of less common genetic diseases such as spino-muscular atrophy (SMA). A variation of this approach is used to induce skipping of a mutated constitutive exon in Duchenne's Muscular Dystrophy (DMD) and thereby restore the reading frame, described as exon re-framing

Myosin phosphatase has been studied as a key nexus where signals converge to regulate vascular smooth muscle tone and thus blood flow and pressure. A thirty-one nucleotide alternative exon was identified within the myosin phosphatase regulatory subunit (Mypt1=PPP1R12a) that by variable coding of a C-terminal leucine zipper motif (LZ) determines the ability of the enzyme to bind cGMP-dependent protein kinase (cGK1a) and thus be activated by NO vasodilator signaling. Using germline (Cre-Lox) modified mice, suppression of splicing of Mypt1 E24 sensitizes VSM to NO/cGMP vasodilator signaling and has a favorable effect on blood pressure.

This background information is provided for informational purposes only. No admission is necessarily intended, nor should it be construed, that any of the preceding information constitutes prior art against the present invention.

### SUMMARY OF THE INVENTION

It is to be understood that both the foregoing general description of the embodiments and the following detailed description are exemplary, and thus do not restrict the scope of the embodiments.

A great weakness and difficulty of gene targeted therapies has been efficient delivery to the required cell type. While vectors and approaches have been tested and optimized for delivery to cardiac and skeletal muscle, liver, and other cell types, little attention has been paid to the smooth muscle cells dispersed throughout the vascular and visceral systems of the body. The few reports that have systematically studied this question have generally reported low efficiency. A strategy targeting Mypt1 E24 to improve vascular function would have to target the smooth muscle cells of the micro-circulation of which there has been little study. In this disclosure, two approaches targeting Mypt1 E24 for therapeutic gain were studied: 1) Crispr/Cas9 editing in vitro followed by viral gene delivery in vivo 2) and anti-sense oligonucleotide injections in vivo.

The present invention relates to compositions and methods for targeting of a splice variant of the regulatory subunit of myosin phosphatase for therapeutic gain in subjects having hypertension and/or heart failure. In some embodiments, the composition comprises a splice blocking anti-sense oligomer, which is a long lasting injectable modified oligonucleotide with a powerful and unique mechanism for treating hypertension and heart failure with preserved ejection fraction (HFpEF).

In one aspect the invention provides an oligomer capable of binding to a target site of precursor messenger RNA (pre-mRNA) of myosin phosphatase target subunit (Mypt1) in cells, wherein binding of the oligomer to the target site of the pre-mRNA suppresses splicing of exon 24 of myosin phosphatase target subunit (Mypt1) in cells.

In another aspect, the invention provides a pharmaceutical composition comprising an effective amount of an oligomer and a pharmaceutically acceptable carrier, wherein the oligomer is capable of binding to a target site of precursor messenger RNA (pre-mRNA) of myosin phosphatase target subunit (Mypt1) in cells, wherein binding of the oligomer to the target site of the pre-mRNA suppresses splicing of exon 24 of myosin phosphatase target subunit (Mypt1) in cells.

In another aspect, the invention provides a method of lowering blood pressure in a subject, comprising administering to the subject an effective amount of an oligomer, wherein the oligomer binds to a target site of precursor messenger RNA (pre-mRNA) of myosin phosphatase target subunit (Mypt1) in cells of the subject, wherein the binding of the oligomer to the target site of the pre-mRNA suppresses splicing of exon 24 of myosin phosphatase target subunit (Mypt1) in the cells, thereby lowering blood pressure in the subject.

In another aspect, the invention provides a method of treating hypertension in a subject, comprising administering to the subject an effective amount of an oligomer, wherein the oligomer binds to a target site of precursor messenger RNA (pre-mRNA) of myosin phosphatase target subunit (Mypt1) in cells of the subject, wherein the binding of the oligomer to the target site of the pre-mRNA suppresses splicing of exon 24 of myosin phosphatase target subunit (Mypt1) in the cells, thereby treating hypertension in the subject.

In another aspect, the invention provides a method of treating heart failure in a subject, comprising administering to the subject an effective amount of an oligomer, wherein the oligomer binds to a target site of precursor messenger RNA (pre-mRNA) of myosin phosphatase target subunit (Mypt1) in cells of the subject, wherein the binding of the oligomer to the target site of the pre-mRNA suppresses splicing of exon 24 of myosin phosphatase target subunit (Mypt1) in the cells, thereby treating heart failure in the subject.

In some embodiments, the target site of the pre-mRNA comprises any sequence within exon 24 and/or flanking intron sequence that when bound to the oligomer suppresses splicing of exon 24. In some embodiments, the target site of the pre-mRNA comprises at least one splice site of exon 24. In some embodiments, the splice site of exon 24 is the 5′ splice site. In some embodiments, the splice site is the 3′ splice site. In some embodiments, the oligomer binds to at least 20 contiguous nucleotides of the pre-mRNA. In some embodiments, the target site comprises at least 20 contiguous nucleotides of SEQ ID NOS: 1, 8 or 9.

In some embodiments, the oligomer comprises a nucleotide sequence that is complementary to the sequence of the target site.

In some embodiments, the oligomer comprises a sequence that is complementary to the target site except at up to two base positions, wherein the oligomer binds to the target site and suppresses splicing of exon 24.

In some embodiments, the oligomer is an oligonucleotide. In some embodiments, the oligonucleotide comprises a DNA or an RNA molecule. In some embodiments, the oligomer comprises a phosphorodiamidate morpholino oligonucleotide (PMO) or a phosphorothioate-linked 2′-O-methyl oligonucleotide (2′OMePS).

In some embodiments, the oligomer suppresses splicing of exon 24 by at least 50%. In some embodiments, the oligomer suppresses splicing of exon 24 by at least 90%.

In some embodiments, the oligomer is between 12 and 40 bases in length. In some embodiments, the oligomer is 25 bases in length.

In some embodiments, the oligomer comprises a base sequence of SEQ ID NO:2 or SEQ ID NO:10.

In some embodiments, the oligomer is conjugated to or complexed with a chemical moiety. In some embodiments, the oligomer is conjugated to octo-guanidine. In some embodiments, the oligomer is a phosphorodiamidate morpholino oligonucleotide comprising a base sequence of SEQ ID NO:2 or SEQ ID NO:10, and conjugated or complexed at the 3′ end to an octo-guanidine.

In some embodiments, the method further comprises administering to the subject an effective amount of one or more treatments for lowering blood pressure. In some embodiments, the one or more treatments comprises administering an agent that lowers blood pressure. In some embodiments, the agent is selected from the group consisting of a diuretic, beta-blocker, alpha-blocker, alpha/beta blocker, centrally acting sympatholytic, peripherally acting sympatholytic, calcium-channel blocker, dihydropyridine, direct vasodilator, angiotensin-converting enzyme (ACE) inhibitor, and combinations thereof.

In some embodiments, the method results in a decreased incidence or probability of heart disease in the subject.

In some embodiments, the method results in a decreased incidence or probability of a heart attack in the subject.

In some embodiments, the method results in a decreased incidence or probability of a stroke in the subject.

In some embodiments, the oligomer is administered at a dose of at least 3 mg/kg.

In some embodiments, the oligomer is administered at a dose of about 6.25 mg/kg. In some embodiments, the oligomer is administered at a dose of about 12.5 mg/kg.

In some embodiments, the oligomer is administered either intravenously, intraperitoneally or subcutaneously. In some embodiments, one to ten doses of oligomer are administered. In some embodiments, a single dose is administered every one to about every 180 days. In some embodiments, a single dose is administered about every 90 to about every 180 days.

In some embodiments, the heart failure is heart failure with preserved Ejection Fraction (HFpEF).

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE FIGURES

The skilled artisan will understand that the drawings, described below, are for illustration purposes only. The drawings are not intended to limit the scope of the present teachings in any way.

FIG. 1. Crispr/Cas9 mediated editing of Mypt1 E24 in vitro. A) Sequence of the target region flanking the exon 24 of PPP1R12A gene. The exon 24 of PPP1R12A is identified by a red box. Five sgRNAs (sgRNA5, sgRNA6, sgRNA9, sgRNA11 and sgRNA12) selected for in vitro evaluation are represented by grey pentagones positioned at the level of their target region on the genomic PPP1R12A sequence. sgRNA5 and sgRNA6 are located upstream the exon 24 of PPP1R12A gene with sgRNA6 overlapping the 3′ end of the exon's splicing site. sgRNA9, sgRNA11 and sgRNA12 are located downstream the exon. All sgRNAs were evaluated individually and in combination with other sgRNAs located on the opposite side of the exon (sgRNAs 5+9, sgRNAs 5+11, sgRNAs 5+12, sgRNAs 6+9, sgRNAs 6+11, sgRNAs 6+12). B. Genomic PCR targeting the PPP1R12A exon 24 flanking region amplifies a 754pb-DNA fragment (control). sgRNA-px601 constructs tested individually yielded genomic edits that were below the resolution of the 2% agarose gel. C. Surveyor nuclease yielded products of 250 bp and 500 bp after cleavage of heteroduplexes generated by hybridization of control DNA and DNA edited by sgRNA6-px601. Similarly, products of 320 bp and 430 bp, 120 bp and 630 bp, 330 bp and 420 bp, 270 bp and 480 bp were observed for DNA edited by sgRNA11-px601, sgRNA5-px601, sgRNA9-px601 and sgRNA12-px601, respectively. As expected, for each experimental condition, the sum of the sizes of both nuclease products was equal to the size of the control DNA (~750 bp). Only 1 product was observed for control DNA treated by surveyor endonuclease, reflecting the absence of mutations. D. sgRNA-px601 6+11, 6+9, 6+12, 5+11, 5+9 and 5+12 duets yielded genomic edits which sizes were approximately 500 bp, 600 bp, 470 bp, 450pb, 570 bp and 420 bp, respectively with a range of efficiencies extending up to ~40% (sgRNAs 6+11 and sgRNAs 5+11).

FIG. 2. An antisense oligonucleotide targeting the 5′ splice site causes effective and long-lasting suppression of Mypt1 E24 splicing. A) The target sequence for the spice-blocking anti-sense oligonucleotide is shown. SBASOE24, or scrambled ASO or vehicle (saline) was injected intra-peritoneally at the doses as shown. Effect on splicing of Mypt1 E24 in mesenteric arteries was measured with a RT-PCR assay with primers flanking the alternative followed by separation of PCR products by 2.5% agarose gel electrophoresis. Percent splicing inclusion was quantified by dividing the E24+ band by the total of the E24+ and E24-bands. The top-post band is artifactual and was excluded from this analysis. B) Dose-response: Mice were injected at the indicated dose every other day for three doses and assayed the following day. A dose of 12.5 mg/kg nearly completely suppressed splicing of Mypt1 E24 with an EC₅₀ of 6.25 mg/kg·n=2-5. ANOVA followed by Dunnett's multiple comparation test. * p<0.05 C) Kinetic: Suppression of Mypt1 E24 splicing in mesenteric arteries was maintained for twenty-eight days after three IP injections every other day of SBASOE24 at 12.5 mg/kg·n=3-5 followed by Dunnett's multiple comparation test. * p<0.05. Abbreviations: SBASO, splice blocking anti-sense oligonucleotide; ss, splice site; d, day; RT, reverse transcriptase;

FIG. 3. MyoNOsen is a 25-mer splice blocking anti-sense oligonucleotide. By binding to the 5′ splice site of Exon 24 of Mypt1, MyoNOsen favors a natural splice variant of Mypt1 mRNA. It encodes the Mypt1/E24-protein isoform, which includes a leucine zipper (LZ) motif. This motif dimerizes with the LZ motif of cGMP-dependent protein kinase a (PKG1 a). The LZ/LZ binding enables Nitric Oxide (NO) signals to activate myosin phosphatase.

FIG. 4. MyoNOsen suppresses splicing of Mypt1 Exon 24. For dose-response study (left), mice were injected IP on days −4, −2, and O with scrambled (scr) oligo or MyoNOsen. At 24 hr, RNA from mesenteric arteries was purified and RT-PCR amplified fragments were run on agarose gels. For kinetics study (right), mice were injected with 12.5 mg/kg MyoNOsen as described. N=3-5 mice/group, representatives shown. p<0.05 vs control.

FIG. 5. MyoNOsen suppresses Angiotensin II-induced hypertension. 8 to 12 wk old female mice were treated with MyoNOsen (blue) vs Control (red: scrambled oligo or Saline. 12.5 mg/kg oligo was i.p injected 3x/wk. Mini-pumps were implanted to deliver 490 ng/kg/min of AngII. Systolic pressure was continuously recorded; 12-hour averaged (Day & Night) systolic pressures are shown (5-6 mice/group).

FIG. 6. Timeline for testing MyoNOsen in HTN.

FIG. 7. Timeline for testing MyoNOsen in HFpEF.

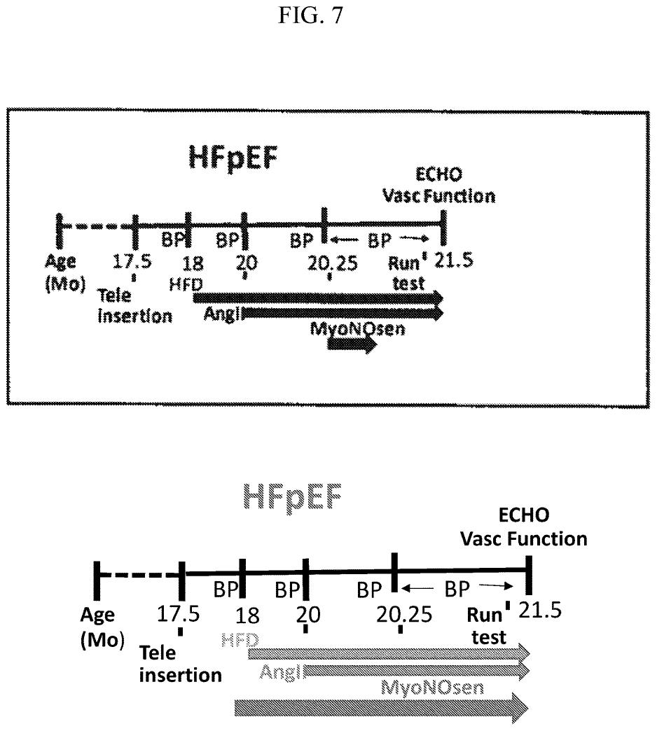

FIG. 8. Schematic of differential splicing and the protein products produced and downstream effects.

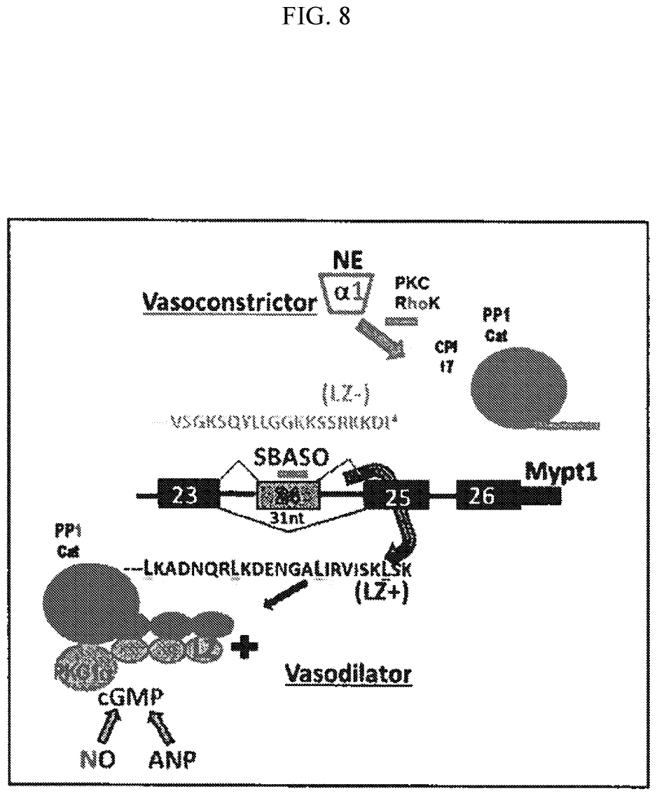

FIG. 9. A. Schematic of MyoNOsen binding to target sequence. B. Schematic of differential splicing and the protein products produced and downstream effects.

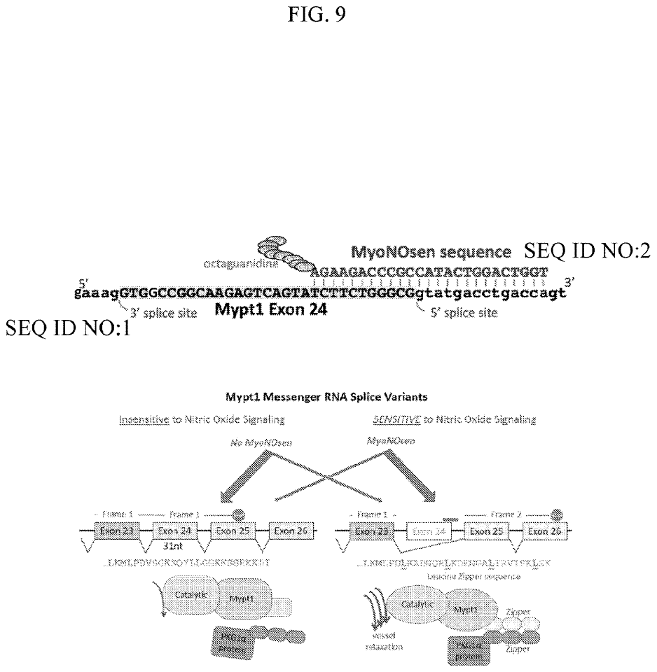

FIG. 10. A. Structure of octo-guanidine conjugate. B. Phosphorodiamidate linkage of oligonucleotide bases (B; n=20-25).

### DETAILED DESCRIPTION OF THE INVENTION

Reference will now be made in detail to the presently preferred embodiments of the invention which, together with the drawings and the following examples, serve to explain the principles of the invention. These embodiments describe in sufficient detail to enable those skilled in the art to practice the invention, and it is understood that other embodiments may be utilized, and that structural, biological, and chemical changes may be made without departing from the spirit and scope of the present invention. Unless defined otherwise, all technical and scientific terms used herein have the same meanings as commonly understood by one of ordinary skill in the art.

The practice of the present invention employs, unless otherwise indicated, conventional techniques of molecular biology (including recombinant techniques), microbiology, cell biology, biochemistry and immunology, which are within the skill of the art. Such techniques are explained fully in the literature. See, e.g., Sambrook et al. Molecular Cloning: A Laboratory Manual, 2^{nd} edition (1989); Current Protocols in Molecular Biology (F. M. Ausubel et al. eds. (1987)); the series Methods in Enzymology (Academic Press, Inc.); PCR: A Practical Approach (M. MacPherson et al. IRL Press at Oxford University Press (1991)); PCR 2: A Practical Approach (M. J. MacPherson, B. D. Hames and G. R. Taylor eds. (1995)); Antibodies, A Laboratory Manual (Harlow and Lane eds. (1988)); Using Antibodies, A Laboratory Manual (Harlow and Lane eds. (1999)); and Animal Cell Culture (R. I. Freshney ed. (1987)).

Definitions of common terms in molecular biology may be found, for example, in Benjamin Lewin, Genes VII, published by Oxford University Press, 2000 (ISBN 019879276X); Kendrew et al. (eds.); The Encyclopedia of Molecular Biology, published by Blackwell Publishers, 1994 (ISBN 0632021829); and Robert A. Meyers (ed.), Molecular Biology and Biotechnology: a Comprehensive Desk Reference, published by Wiley, John & Sons, Inc., 1995 (ISBN 0471186341).

For the purpose of interpreting this specification, the following definitions will apply and whenever appropriate, terms used in the singular will also include the plural and vice versa. In the event that any definition set forth below conflicts with the usage of that word in any other document, including any document incorporated herein by reference, the definition set forth below shall always control for purposes of interpreting this specification and its associated claims unless a contrary meaning is clearly intended (for example in the document where the term is originally used). The use of “or” means “and/or” unless stated otherwise. As used in the specification and claims, the singular form “a,” “an” and “the” include plural references unless the context clearly dictates otherwise. For example, the term “a cell” includes a plurality of cells, including mixtures thereof. The use of “comprise,” “comprises,” “comprising,” “include,” “includes,” and “including” are interchangeable and not intended to be limiting. Furthermore, where the description of one or more embodiments uses the term “comprising,” those skilled in the art would understand that, in some specific instances, the embodiment or embodiments can be alternatively described using the language “consisting essentially of” and/or “consisting of.”

As used herein, the term “about” means plus or minus 10% of the numerical value of the number with which it is being used.

The terms “nucleic acid,” and “polynucleotide,” are used interchangeably and refer to a deoxyribonucleotide or ribonucleotide polymer, in linear or circular conformation, and in either single- or double-stranded form. For the purposes of the present disclosure, these terms are not to be construed as limiting with respect to the length of a polymer. The terms can also encompass analogues of natural nucleotides, as well as nucleotides that are modified in the base, sugar and/or phosphate moieties.

The terms “polypeptide,” “peptide” and “protein” are used interchangeably to refer to a polymer of amino acid residues. The term also applies to amino acid polymers in which one or more amino acids are chemical analogues or modified derivatives of corresponding naturally-occurring amino acids. The term “sequence” relates to a nucleotide sequence of any length, which can be DNA or RNA; can be linear, circular or branched and can be either single-stranded or double stranded.

The term “identity” relates to an exact nucleotide-to-nucleotide or amino acid-to-amino acid correspondence of two polynucleotides or polypeptide sequences, respectively. Two or more sequences (polynucleotide or amino acid) can be compared by determining their percent identity. Calculations of homology or sequence identity between two sequences (the terms are used interchangeably herein) are performed as follows. The sequences are aligned for optimal comparison purposes (e.g., gaps can be introduced in one or both of a first and a second amino acid or nucleic acid sequence for optimal alignment and non-homologous sequences can be disregarded for comparison purposes). The optimal alignment is determined as the best score using the GAP program in the GCG software package with a Blossum 62 scoring matrix with a gap penalty of 12, a gap extend penalty of 4, and a frame shift gap penalty of 5. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences.

“Sequence similarity” between polynucleotides can be determined by hybridization of polynucleotides under conditions that allow formation of stable duplexes between homologous regions, followed by digestion with single-stranded-specific nuclease(s), and size determination of the digested fragments.

“Subject,” as used herein, may mean either a human or non-human animal. The term includes, but is not limited to, mammals (e.g., humans, other primates, pigs, rodents (e.g., mice and rats or hamsters), rabbits, guinea pigs, cows, horses, cats, dogs, sheep, and goats). In one embodiment, the subject is a human. In some embodiments, the subject is a mouse.

The term “therapeutically effective amount” means the total amount of each active component of the pharmaceutical composition or method that is sufficient to show meaningful patient benefits, i.e, a decrease in the subject's blood pressure.

“Treatment” of an individual (e.g. a mammal, such as a human) or a cell is any type of intervention used in an attempt to alter the natural course of the individual or cell. Treatment includes, but is not limited to, administration of a pharmaceutical composition, and may be performed either prophylactically or subsequent to the initiation of a pathologic event or contact with an etiologic agent. Treatment includes any desirable effect on the symptoms or pathology of a disease or condition associated with the high blood pressure, heart failure, heart disease, or conditions associated with the Mypt1 protein, and may include, for example, minimal changes or improvements in one or more measurable effects of the disease or condition being treated. Also included are “prophylactic” treatments, which can be directed to reducing the rate of progression of the disease or condition being treated, delaying the onset of that disease or condition, or reducing the severity of its onset. “Treatment” or “prophylaxis” does not necessarily indicate complete eradication, cure, or prevention of the disease or condition, or associated symptoms thereof.

The inventors discovered that antisense oligomers suppress splicing of Mypt1 E24. The data presented herein constitute a proof-of-principle that oligomer-mediated antisense suppression of exon 24 leads to physiologically significant effects of lowering blood pressure, treating hypertension or heart failure.

### Oligomers and Methods of Treatment

The present invention relates to oligomers which can bind to pre-mRNA produced from the Mypt1 gene and cause suppression of splicing of Mypt1 exon 24 during cellular processing of the pre-mRNA.

In some embodiments, the invention provides an oligomer capable of binding to a target site of precursor messenger RNA (pre-mRNA) of myosin phosphatase target subunit (Mypt1) in cells, wherein binding of the oligomer to the target site of the pre-mRNA suppresses splicing of exon 24 of myosin phosphatase target subunit (Mypt1) in cells.

In another embodiment, the invention provides a method of lowering blood pressure in a subject, comprising administering to the subject an effective amount of an oligomer, wherein the oligomer binds to a target site of precursor messenger RNA (pre-mRNA) of myosin phosphatase target subunit (Mypt1) in cells of the subject, wherein the binding of the oligomer to the target site of the pre-mRNA suppresses splicing of exon 24 of myosin phosphatase target subunit (Mypt1) in the cells, thereby lowering blood pressure in the subject.

In another embodiment, the invention provides a method of treating hypertension in a subject, comprising administering to the subject an effective amount of an oligomer, wherein the oligomer binds to a target site of precursor messenger RNA (pre-mRNA) of myosin phosphatase target subunit (Mypt1) in cells of the subject, wherein the binding of the oligomer to the target site of the pre-mRNA suppresses splicing of exon 24 of myosin phosphatase target subunit (Mypt1) in the cells, thereby treating hypertension in the subject.

In another embodiment, the invention provides a method of treating heart failure in a subject, comprising administering to the subject an effective amount of an oligomer, wherein the oligomer binds to a target site of precursor messenger RNA (pre-mRNA) of myosin phosphatase target subunit (Mypt1) in cells of the subject, wherein the binding of the oligomer to the target site of the pre-mRNA suppresses splicing of exon 24 of myosin phosphatase target subunit (Mypt1) in the cells, thereby treating heart failure in the subject. In some embodiments, the heart failure is heart failure with preserved Ejection Fraction (HFpEF).

Isoforms of the myosin phosphatase regulatory subunit (Mypt1) are generated by alternative splicing of a 31 nucleotide exon 24. In some embodiments, the splicing of Mypt1 exon 24 is suppressed by administration of the oligomer in cells of the subject. In some embodiments, human exon 24 is represented by SEQ ID NO:11 and mouse exon 24 is represented by SEQ ID NO:3.

In some embodiments, the subject has a systolic blood pressure prior to treatment of at least 140 mm Hg. In some embodiments, the subject has a systolic blood pressure prior to treatment of at least 160 mm Hg. In some embodiments, the subject has a diastolic blood pressure prior to treatment of at least 90 mm Hg. In some embodiments, the subject has a diastolic blood pressure prior to treatment of at least 100 mm Hg. In some embodiments, the subject has a systolic blood pressure prior to treatment of at least 160 mm Hg and a diastolic blood pressure of at least 100 mm Hg. In some embodiments, the subject's systolic blood pressure can be lowered by at least about 5 mm Hg, by at least about 10 mm Hg, by at least about 15 mm Hg, by at least about 20 mm Hg, by at least about 25 mm Hg, by at least about 30 mm Hg, by at least about 35 mm Hg, by at least about 40 mm Hg, by at least about 45 mm Hg, or by at least about 50 mm Hg. In some embodiments, the subject's diastolic blood pressure can be lowered by at least about 2 mm Hg, by at least about 5 mm Hg, by at least about 10 mm Hg, by at least about 15 mm Hg, by at least about 20 mm Hg, by at least about 25 mm Hg, by at least about 30 mm Hg, by at least about 35 mm Hg, or by at least about 40 mm Hg.

A “target site” or “target sequence” is a nucleic acid sequence that defines a general region of a nucleic acid to which a binding molecule may bind, in this case, an antisense oligomer, provided sufficient conditions for binding exist.

In some embodiments, the oligomer is fully complementary to, and binds to the target site in an antisense fashion.

In some embodiments, the region on human Mypt1 targeted for suppression of splicing of exon 24 is within 155881 bp to 156420 bp (SEQ ID NO:4) of the human Mypt1 gene (PPP1R12A) (gene sequence ID ENSG00000058272) or pre-mRNA. This region corresponds to a 540 bp-long region containing exon 24 and flanking intronic sequence. In some embodiments, the target site comprises a sequence that falls within 120 nucleotides upstream to 200 nucleotides downstream of exon 24. Thus, in some embodiments, the target site comprises a sequence that falls within these 351 nucleotides, which corresponds to SEQ ID NO:8, for example, a span of 12-40, 20-40, 25-40 or at least 20 or 25 contiguous nucleotides of SEQ ID NO:8.

The term “oligomer” or “oligonucleotide” in the context of the present invention refers to a molecule (i.e., an oligonucleotide) formed by the covalent linkage of two or more nucleotides. In this specification, a single nucleotide (unit) may be referred to as a monomer or a unit. In some embodiments, the terms “nucleoside,” “nucleotide,” “base,” and “nucleobase” are used interchangeably. When referring to a nucleotide or base sequence, it will be appreciated that what is referred to is a sequence of bases such as A, T, G, C, or U.

The terms “antisense oligomer” and “antisense compound” and “antisense oligonucleotide” are used interchangeably and refer to a sequence of cyclic subunits, each bearing a base-pairing moiety, linked by intersubunit linkages that allow the base-pairing moieties to hybridize to a target sequence in a nucleic acid (typically an RNA) by Watson-Crick base pairing, to form a nucleic acid: oligomer heteroduplex within the target sequence. The cyclic subunits are based on ribose or another pentose sugar or, in a preferred embodiment, a morpholino group (see description of morpholino oligomers below). The oligomer may have exact or near sequence complementarity to the target sequence; variations in sequence near the termini of an oligomer are generally preferable to variations in the interior.

Such an antisense oligomers can be designed to inhibit natural pre-mRNA splice processing, and may be said to be “directed to” or “targeted against” a target sequence with which it hybridizes. The target sequence is typically a region including a splice site of a pre-processed mRNA. In some embodiments, the target sequence for a splice site may include an mRNA sequence having its 5′ end 1 to about 25 base pairs downstream of a normal splice acceptor junction in a preprocessed mRNA. A preferred target sequence is any region of a preprocessed mRNA that includes a splice site or is contained entirely within an exon coding sequence or spans a splice acceptor or donor site.

An “exon” refers to a defined section of nucleic acid that encodes for a protein, or a nucleic acid sequence that is represented in the mature form of an RNA molecule after either portions of a pre-processed (or precursor) RNA have been removed by splicing. The mature RNA molecule can be a messenger RNA (mRNA).

“Exon skipping” refers generally to the process by which an entire exon, or a portion thereof, is removed from a given pre-processed RNA, and is thereby excluded from being present in the mature RNA, such as the mature mRNA that is translated into a protein. Hence, the portion of the protein that is otherwise encoded by the skipped exon is not present in the expressed form of the protein, typically creating an altered, though still functional, form of the protein. In certain embodiments, the exon being skipped is Exon 24 from the human Mypt1 gene.

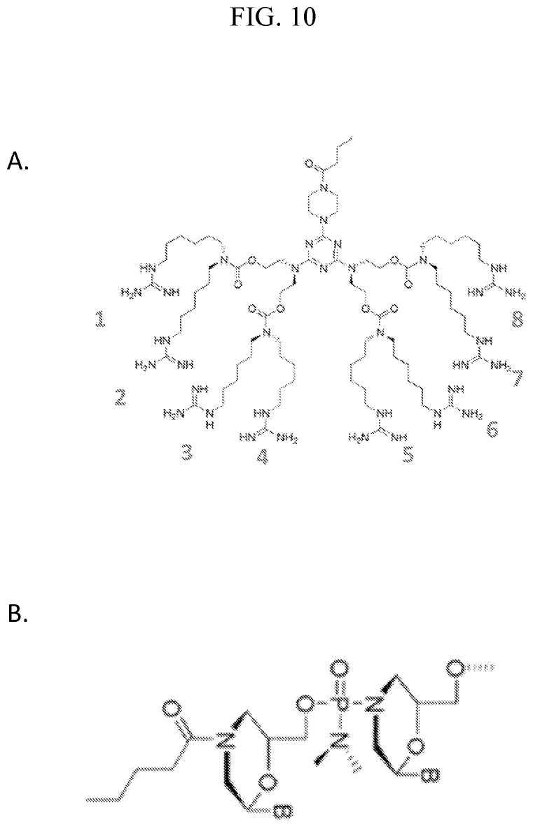

The terms “morpholino oligomer” or “PMO” (phosphoramidate-or phosphorodiamidate morpholino oligomer) refer to an oligonucleotide analog composed of morpholino subunit structures, where (i) the structures are linked together by phosphorus-containing linkages, one to three atoms long, preferably two atoms long, and preferably uncharged or cationic, joining the morpholino nitrogen of one subunit to a 5′ exocyclic carbon of an adjacent subunit, and (ii) each morpholino ring bears a purine or pyrimidine base-pairing moiety effective to bind, by base specific hydrogen bonding, to a base in a polynucleotide. See, for example, the structure in FIG. 10, which shows a preferred phosphorodiamidate linkage type. Variations can be made to this linkage as long as they do not interfere with binding or activity. For example, the oxygen attached to phosphorus may be substituted with sulfur (thiophosphorodiamidate). The 5′ oxygen may be substituted with amino or lower alkyl substituted amino. The pendant nitrogen attached to phosphorus may be unsubstituted, monosubstituted, or disubstituted with (optionally substituted) lower alkyl. The purine or pyrimidine base pairing moiety is typically adenine, cytosine, guanine, uracil, thymine or inosine. The synthesis, structures, and binding characteristics of morpholino oligomers are detailed in U.S. Pat. Nos. 5,698,685, 5,217,866, 5,142,047, 5,034,506, 5,166,315, 5,521,063, 5,506,337, 8,076,476, 8,299,206 and 7,943,762 (cationic linkages), all of which are incorporated herein by reference. Modified intersubunit linkages and terminal groups are detailed in PCT application US2011/038459 and publication WO/2011/150408 which are incorporated herein by reference in their entirety.

The terms “complementary” and “complementarity” refer to polynucleotides (i.e., a sequence of nucleotides) related by base-pairing rules. Complementarity may be “partial,” in which only some of the nucleic acids' bases are matched according to base pairing rules. Or, there may be “complete” or “total” complementarity between the nucleic acids. The degree of complementarity between nucleic acid strands has significant effects on the efficiency and strength of hybridization between nucleic acid strands. While perfect complementarity is often desired, some embodiments can include one or more but preferably 6,5,4, 3, 2, or 1 mismatches with respect to the target RNA. Variations at any location within the oligomer are included. In certain embodiments, variations in sequence near the termini of an oligomer are generally preferable to variations in the interior, and if present are typically within about 6, 5, 4, 3, 2, or 1 nucleotides of the 5′ and/or 3′ terminus.

In some embodiments, the target site of the pre-mRNA comprises any sequence within exon 24 and/or flanking intron sequence that when bound to the oligomer suppresses splicing of exon 24. In some embodiments, the target site of the pre-mRNA comprises a splice site of exon 24. In some embodiments, the splice site of exon 24 is the 5′ splice site. In some embodiments, the splice site is the 3′ splice site. In some embodiments, the target site comprises both the 3′ and 5′ splice sites. In some embodiments, the oligomer binds to at least 12-40 contiguous nucleotides of the pre-mRNA. In some embodiments, the target site comprises at least 20 contiguous nucleotides of SEQ ID NO:1, SEQ ID NO:8, or SEQ ID NO: 9.

The oligomer can be any type of oligomer as long as it has the selected base sequence and can bind to a target site of the Mypt1 pre-mRNA to cause exon skipping of exon 24. For example, the oligomer can be an oligodeoxyribonucleotide, an oligoribonucleotide, a phosphorodiamidate morpholino oligonucleotide (PMO) or a phosphorothioate-linked 2′-O-methyl oligonucleotide (2′OMePS). Preferably, the oligomer is a PMO or a 2′OMePS. In one embodiment, the oligomer is a PMO. The advantage of a PMO is that it has excellent safety profiles and appears to have longer lasting effects in vivo compared to 2′OMePS oligonucleotides. Preferably, the oligomer is isolated so that it is free from other compounds or contaminants.

In some embodiments, the base sequence of the oligomer can vary from the selected sequence at up to two base positions. If the base sequence does vary at two positions, the oligomer will still be able to bind to the Mypt1 pre-mRNA to cause exon skipping. Preferably, the base sequence of the oligomer varies from the selected sequence at up to one base position and, more preferably, the base sequence does not vary from the selected sequence. The less that the base sequence of the oligomer varies from the selected sequence, the more efficiently it binds to the target site in order to cause exon skipping. In some embodiments, the oligomer comprises a nucleotide sequence that is complementary to the sequence of the target site.

Without being restricted to any particular theory, it is thought that the binding of the oligomers to the Mypt1 pre-mRNA interacts with or interferes with the binding of SR proteins to the exon. SR proteins are involved in the splicing process of adjacent exons. Therefore, it is thought that interacting or interfering with the binding of the SR proteins interferes with the splicing machinery resulting in exon skipping.

In some embodiments, the oligomer is at least 20 bases in length. Preferably, the oligomer is at least 25 bases in length. In some embodiments, the oligomer may be at least 28 bases in length or at least 30 bases in length. Preferably, the oligomer is no more than 40 bases in length. In some embodiments, the oligomer may be no more than 35 bases in length or no more than 32 bases in length. Preferably, the oligomer is between 20 and 40 bases in length. More preferably, the oligomer is between 25 and 35 bases in length. In some embodiments, the oligomer is between 28 and 32 bases in length, between 29 and 31 bases in length, or about 30 bases in length. It has been found that an oligomer which is 30 bases in length causes efficient exon skipping. If the oligomer is longer than 40 bases in length, the specificity of the binding to the target site may be reduced. If the oligomer is less than 20 bases in length, the exon skipping efficiency may be reduced.

In some embodiments, the oligomer comprises a sequence that is complementary to the target site except at up to two base positions, wherein the oligomer binds to the target site and suppresses splicing of exon 24.

In some embodiments, the oligomer has full complementarity with the target sequence. In some embodiments, the targeting domain has or includes 1, 2, 3, 4, 5, 6, 7 or 8 nucleotides that are not complementary with the corresponding nucleotide of the targeting domain.

In some embodiments, the oligomer includes 1, 2, 3, 4 or 5 nucleotides that are complementary with the corresponding nucleotide of the target sequence within 5 nucleotides of its 5′ end. In an embodiment, the oligomer includes 1, 2, 3, 4 or 5 nucleotides that are complementary with the corresponding nucleotide of the target sequence within 5 nucleotides of its 3′ end.

In some embodiments, the oligomer includes 1, 2, 3, or 4 nucleotides that are not complementary with the corresponding nucleotide of the target sequence within 5 nucleotides of its 5′ end. In some embodiments, the oligomer includes 1, 2, 3, or 4 nucleotides that are not complementary with the corresponding nucleotide of the target sequence within 5 nucleotides of its 3′ end.

In some embodiments, the degree of complementarity, together with other properties of the oligomer, is sufficient to allow targeting of a oligomer to the target site of the Mypt1 pre-mRNA.

In some embodiments, the oligomer comprises two consecutive nucleotides that are not complementary to the target site (“non-complementary nucleotides”), e.g., two consecutive noncomplementary nucleotides that are within 5 nucleotides of the 5′ end of the target site, within 5 nucleotides of the 3′ end of the target site, or more than 5 nucleotides away from one or both ends of the target site.

In some embodiments, no two consecutive nucleotides within 5 nucleotides of the 5′ end of the target site, within 5 nucleotides of the 3′ end of the target site, or within a region that is more than 5 nucleotides away from one or both ends of the target site, are not complementary to the target site.

In some embodiments, there are no noncomplementary nucleotides within 5 nucleotides of the 5′ end of the target site, within 5 nucleotides of the 3′ end of the target site, or within a region that is more than 5′ nucleotides away from one or both ends of the target site.

In some embodiments, the oligomer is an oligonucleotide. In some embodiments, the oligomer is a modified oligonucleotide. In some embodiments, the oligonucleotide comprises a DNA or an RNA molecule. In some embodiments, the oligomer is a phosphorodiamidate morpholino oligonucleotide (PMO) or a phosphorothioate-linked 2′-O-methyl oligonucleotide (2′OMePS).

In some embodiments, the antisense oligomer comprises a backbone modification comprising a phosphorothioate linkage or a phosphorodiamidate linkage. In some embodiments, the antisense oligomer comprises a phosphorodiamidate morpholino, a locked nucleic acid, a peptide nucleic acid, a 2′-O-methyl, a 2′-Fluoro, or a 2′-O-methoxyethyl moiety.

In some embodiments, the antisense oligomer comprises at least one modified sugar moiety. In some embodiments, each sugar moiety is a modified sugar moiety. In some embodiments, the antisense oligomer consists of from 8 to 50 nucleobases, 8 to 40 nucleobases, 8 to 35 nucleobases, 8 to 30 nucleobases, 8 to 25 nucleobases, 8 to 20 nucleobases, 8 to 15 nucleobases, 9 to 50 nucleobases, 9 to 40 nucleobases, 9 to 35 nucleobases, 9 to 30 nucleobases, 9 to 25 nucleobases, 9 to 20 nucleobases, 9 to 15 nucleobases, 10 to 50 nucleobases, 10 to 40 nucleobases, 10 to 35 nucleobases, 10 to 30 nucleobases, 10 to 25 nucleobases, 10 to 20 nucleobases, 10 to 15 nucleobases, 11 to 50 nucleobases, 11 to 40 nucleobases, 11 to 35 nucleobases, 11 to 30 nucleobases, 11 to 25 nucleobases, 11 to 20 nucleobases, 11 to 15 nucleobases, 12 to 50 nucleobases, 12 to 40 nucleobases, 12 to 35 nucleobases, 12 to 30 nucleobases, 12 to 25 nucleobases, 12 to 20 nucleobases, or 12 to 15 nucleobases.

In some embodiments, the antisense oligomer is at least 80%, at least 85%, at least 90%, at least 95%, at least 98%, at least 99%, or 100%, complementary to the target site of the Mypt1 pre-mRNA. In some embodiments, the target site of the Mypt1 pre-mRNA is within a sequence of SEQ ID NO:1, SEQ ID NO:8, or SEQ ID NO:9.

In some embodiments, the antisense oligomer comprises a base sequence that is at least about 90% identical to SEQ ID NO:2 or SEQ ID NO:10.

In some embodiments, the oligomer is between 12 and 40 bases in length. In some embodiments, the oligomer is 20-25 bases in length. In some embodiments, the oligomer is 25 bases in length.

In some embodiments, the oligomer comprises a base sequence of SEQ ID NO:2 or SEQ ID NO:10.

In embodiments, the oligomer of the invention are chemically linked to one or more moieties or conjugates, e.g., a targeting moiety or other conjugate that enhances the activity or cellular uptake of the oligomer. Such moieties include, but are not limited to, a lipid moiety, e.g., as a cholesterol moiety, a cholesteryl moiety, an aliphatic chain, e.g., dodecandiol or undecyl residues, a polyamine or a polyethylene glycol chain, or adamantane acetic acid. Oligonucleotides comprising lipophilic moieties, and preparation methods have been described in the published literature. In embodiments, the oligomer is conjugated with a moiety including, but not limited to, an abasic nucleotide, a polyether, a polyamine, a polyamide, a peptides, a carbohydrate, e.g., N-acetylgalactosamine (GalNAc), N—Ac-Glucosamine (GluNAc), or mannose (e.g., mannose-6-phosphate), a lipid, or a polyhydrocarbon compound. Conjugates can be linked to one or more of any nucleotides comprising the oligomer at any of several positions on the sugar, base or phosphate group, as understood in the art and described in the literature, e.g., using a linker. Linkers can include a bivalent or trivalent branched linker. In embodiments, the conjugate is attached to the 3′ end of the oligomer. Methods of preparing oligonucleotide conjugates are described, e.g., in U.S. Pat. No. 8,450,467, “Carbohydrate conjugates as delivery agents for oligonucleotides,” incorporated by reference herein.

In some embodiments, the oligomer may be conjugated to or complexed with a targeting protein in order to target the oligomer to, for example, muscle tissue. If the oligomer is conjugated to an entity, it may be conjugated directly or via a linker. In one embodiment, a plurality of oligomers may be conjugated to or complexed with a single entity. For example, the oligomer may be conjugated to octa-guanidine dendrimers.

Alternatively, an arginine-rich cell penetrating peptide (CPP) can be conjugated to or complexed with the oligomer. In particular, (R-Ahx-R) 4AhxB can be used, where Ahx is 6-aminohexanoic acid and B is beta-alanine (Moulton H M et al. (2007) Biochem. Soc. Trans. 35:826-8), or alternatively (RXRRBR) 2XB can be used. In some embodiments, the oligomer is conjugated to octo-guanidine (see FIG. 10). See, e.g., BioTechniques 2008 Vol. 45 Issue 6, pages 613-623, which is incorporated by reference herein.

In some embodiments, the target site comprises a series of contiguous nucleotides of Mypt1 pre-mRNA that comprises a splice site for exon 24. In some embodiments, the target site comprises a series of contiguous nucleotides of SEQ ID NO:1, SEQ ID NO:8 or SEQ ID NO:9. In some embodiments, the target site comprises at least 12, 13, 14, 15, 16, 17, 18, 19, 20, 21, 22, 23, 24, 25, 26, 27, 28, 29, 30, 31, 32, 33, 34, 35, 36, 37, 38 39 or 40 contiguous nucleotides of SEQ ID NO:1, SEQ ID NO:8 or SEQ ID NO:9.

In some embodiments, oligomers of the disclosure can be a contiguous sequence of nucleotides that is fully or partially complementary to a nucleotide sequence of Mypt1 pre-mRNA that comprises a splice site for exon 24. In some embodiments, oligomers of the disclosure can be a contiguous sequence of 12-40 bases (e.g., morpholino oligomer) or nucleotides that is fully complementary to a corresponding length of SEQ ID NO:1, SEQ ID NO: 8 or SEQ ID NO:9. In some embodiments, the oligomer is not perfectly complementary to SEQ ID NO:1, SEQ ID NO:8 or SEQ ID NO:9, for example, lacking complementarity in one or more nucleotides. In some embodiments, the oligomer differs in one or more nucleotides. In some embodiments, the oligomer comprises 20-30 nucleotides in length.

An “effective amount” or “therapeutically effective amount” refers to an amount of therapeutic compound, such as an antisense oligomer, administered to a human subject, either as a single dose or as part of a series of doses, which is effective to produce a desired therapeutic effect. For an antisense oligonucleotide, this effect is typically brought about by inhibiting translation or natural splice-processing of a selected target sequence. In some embodiments, an effective amount is at least 2 mg/kg of a composition including an antisense oligonucleotide for a period of time to treat the subject. In one embodiment, an effective amount is at least 6.25 mg/kg of a composition including an antisense oligonucleotide to lower blood pressure, treat hypertension or treat heart failure. In another embodiment, an effective amount is at least 12.5 mg/kg of a composition including an antisense oligonucleotide to lower blood pressure, treat hypertension or treat heart failure. In another embodiment, an effective amount is at least 3 mg/kg to about 25 mg/kg, about 25 mg/kg to about 30 mg/kg, or about 30 mg/kg to about 50 mg/kg. In yet another embodiment, an effective amount is about 30 mg/kg or about 50 mg/kg. In another aspect, an effective amount is at least 20 mg/kg, about 25 mg/kg, about 30 mg/kg, or about 30 mg/kg to about 50 mg/kg, for at least 24 weeks, at least 36 weeks, or at least 48 weeks.

In some embodiments, the oligomer is administered at a dose of at least 3 mg/kg. In some embodiments, the oligomer is administered at a dose of about 6.25 mg/kg. In some embodiments, the oligomer is administered at a dose of about 12.5 mg/kg.

In some embodiments, the oligomer is administered either intravenously, intraperitoneally or subcutaneously. In some embodiments, one to ten doses of oligomer are administered. In some embodiments, a single dose is administered every one to about every 180 days. In some embodiments, a single dose is administered about every 30 days, about every 60 days, about every 90 days, about every 120 days, about every 180 days, or longer.

In some embodiments, the subject is a human. In some embodiments, the subject is a non-human animal. In some embodiments, the subject is a fetus, an embryo, or a child. In some embodiments, the cells are ex vivo. In some embodiments, the antisense oligomer is administered by intraperitoneal injection, intramuscular injection, subcutaneous injection, or intravenous injection of the subject.

The advantage provided by the oligomer is that it causes exon skipping, i.e., suppression of splicing of exon 24. Preferably, the oligomer causes an exon skipping rate of at least 40%, i.e. exon 24 will be skipped 40% of the time. More preferably, the oligomer causes an exon skipping rate of at least 50%, more preferably still, at least 60%, even more preferably, at least 70%, more preferably still, at least 75%, more preferably, at least 80%, even more preferably, at least 85%, more preferably still, at least 90%, even most preferably, at least 95%, more preferably, at least 98% and even more preferably, at least about 99%. Exon skipping can be measured by electrophoretic densitometric analysis of RT-PCR reaction products.

In some embodiments, the total amount of target protein produced by the cell contacted with the antisense oligomer is increased about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to the total amount of target protein produced by a control cell, such as a smooth muscle cell.

In some embodiments, the method further comprises administering to the subject an effective amount of one or more treatments for lowering blood pressure.

In some embodiments, the method is combined with administration of a diuretic (e.g., furosemide, hydrochlorothiazide, and spironolactone), beta-blocker (e.g., propranolol, metoprolol, and atenolol), alpha-blocker (e.g., doxazosin, prazosin, and terazosin), alpha/beta blocker (e.g., labetalol and carvedilol), centrally acting sympatholytic (e.g., methyldopa), peripherally acting sympatholytic (e.g., reserpine), calcium-channel blocker (e.g., nifedipine, verapamil, and diltiazem), dihydropyridine (e.g., amlodipine), direct vasodilator (e.g., hydralazine and minoxidil), activators of this pathway (nitric oxide donor drugs such as nitroglycerin, guanylate cyclase activators such as veraciguat, and phosphodiesterase inhibitors such as sildenafil), angiotensin-converting enzyme (ACE) inhibitor (e.g., enalapril, captopril, lisinopril, and benazepril), and combinations thereof.

In some embodiments, the method results in a decreased incidence or probability of heart disease in the subject. In some embodiments, the method results in a decreased incidence or probability of a heart attack in the subject. In some embodiments, the method results in a decreased incidence or probability of a stroke or other adverse vascular event in the subject.

### Vectors and Host Cells

In another aspect, the present invention provides a vector for inducing exon skipping of exon 24 during processing of Mypt1 pre-mRNA, the vector encoding an oligomer of the invention, wherein when the vector is introduced into a cell (e.g. a human cell), the oligomer is expressed. For example, it is possible to express antisense sequences in the form of a gene, which can thus be delivered on a vector. One way to do this would be to modify the sequence of a U7 snRNA gene to include an antisense sequence according to the invention. The U7 gene, complete with its own promoter sequences, can be delivered on an adeno-associated virus (AAV) vector, to induce exon skipping. Similar methods to achieve exon skipping, by using a vector encoding an oligomer of the invention, would be apparent to one skilled in the art.

By “vector” or “nucleic acid construct” is meant a polynucleotide molecule, preferably a DNA molecule derived, for example, from a plasmid, bacteriophage, yeast or virus, into which a polynucleotide can be inserted or cloned. A vector preferably contains one or more unique restriction sites and can be capable of autonomous replication in a defined host cell including a target cell or tissue or a progenitor cell or tissue thereof, or be integrated with the genome of the defined host such that the cloned sequence is reproducible. Accordingly, the vector can be an autonomously replicating vector, i.e., a vector that exists as an extra-chromosomal entity, the replication of which is independent of chromosomal replication, e.g., a linear or closed circular plasmid, an extra-chromosomal element, a mini-chromosome, or an artificial chromosome. The vector can contain any means for assuring self-replication. Alternatively, the vector can be one which, when introduced into the host cell, is integrated into the genome and replicated together with the chromosome(s) into which it has been integrated.

### Pharmaceutical Compositions

The present invention also provides a pharmaceutical composition for inducing exon skipping during processing of Mypt1 pre-mRNA, the composition comprising an oligomer as described above or a vector as described above and a pharmaceutically acceptable carrier, adjuvant or vehicle. Pharmaceutical compositions or formulations comprising the antisense oligonucleotide of the described compositions and for use in any of the described methods can be prepared according to conventional techniques well known in the pharmaceutical industry and described in the published literature. In embodiments, a pharmaceutical composition or formulation for treating a subject comprises an effective amount of any antisense oligomer as described above, or a pharmaceutically acceptable salt, solvate, hydrate or ester thereof, and a pharmaceutically acceptable diluent. The antisense oligomer of a pharmaceutical formulation may further comprise a pharmaceutically acceptable excipient, diluent or carrier.

In some embodiments, the invention provides a pharmaceutical composition comprising an effective amount of an oligomer and a pharmaceutically acceptable carrier, wherein the oligomer is capable of binding to a target site of precursor messenger RNA (pre-mRNA) of myosin phosphatase target subunit (Mypt1) in cells, wherein binding of the oligomer to the target site of the pre-mRNA suppresses splicing of exon 24 of myosin phosphatase target subunit (Mypt1) in cells.

Pharmaceutical compositions of this invention comprise an oligomer of the present invention, and pharmaceutically acceptable salts, esters, salts of such esters, or any other compound which, upon administration to a subject (e.g. a human), is capable of providing (directly or indirectly) the biologically active oligomer thereof, with a pharmaceutically acceptable carrier, adjuvant or vehicle. Pharmaceutically acceptable carriers, adjuvants and vehicles that may be used in the pharmaceutical compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

Pharmaceutically acceptable salts are suitable for use in contact with the tissues of humans and lower animals without undue toxicity, irritation, allergic response, etc., and are commensurate with a reasonable benefit/risk ratio. (See, e.g., S. M. Berge, et al., J. Pharmaceutical Sciences, 66:1-19 (1977), incorporated herein by reference for this purpose. The salts can be prepared in situ during the final isolation and purification of the compounds, or separately by reacting the free base function with a suitable organic acid. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other documented methodologies such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate.

In some embodiments, the compositions are formulated into any of many possible dosage forms such as, but not limited to, tablets, capsules, gel capsules, liquid syrups, soft gels, suppositories, and enemas. In embodiments, the compositions are formulated as suspensions in aqueous, non-aqueous or mixed media. Aqueous suspensions may further contain substances that increase the viscosity of the suspension including, for example, sodium carboxymethylcellulose, sorbitol and/or dextran. The suspension may also contain stabilizers. In embodiments, a pharmaceutical formulation or composition of the present invention includes, but is not limited to, a solution, emulsion, microemulsion, foam or liposome-containing formulation (e.g., cationic or noncationic liposomes).

In some embodiments, a range of nanoparticle systems can be used to deliver the oligomers. In some embodiments, the compositions are administered by intraperitoneal, subcutaneous or intravenous injection.

The pharmaceutical composition or formulation of the present invention may comprise one or more penetration enhancer, carrier, excipients or other active or inactive ingredients as appropriate and well known to those of skill in the art or described in the published literature. In embodiments, liposomes also include sterically stabilized liposomes, e.g., liposomes comprising one or more specialized lipids. These specialized lipids result in liposomes with enhanced circulation lifetimes. In embodiments, a sterically stabilized liposome comprises one or more glycolipids or is derivatized with one or more hydrophilic polymers, such as a polyethylene glycol (PEG) moiety. In embodiments, a surfactant is included in the pharmaceutical formulation or compositions. The use of surfactants in drug products, formulations and emulsions is well known in the art. In embodiments, the present invention employs a penetration enhancer to effect the efficient delivery of the antisense oligonucleotide, e.g., to aid diffusion across cell membranes and/or enhance the permeability of a lipophilic drug. In embodiments, the penetration enhancers is a surfactant, fatty acid, bile salt, chelating agent, or non-chelating nonsurfactant.

Application of the teachings of the present invention to a specific problem is within the capabilities of one having ordinary skill in the art in light of the teaching contained herein. Examples of the compositions and methods of the invention appear in the following non-limiting Examples.

### EXAMPLES

### Example 1. Efficient Suppression of Splicing Smooth Muscle Myosin Phosphatase Alternative Exon 24 with an Anti-Sense Oligonucleotide

The Myosin phosphatase (MP) enzyme is a critical hub upon which signals converge to regulate vessel tone. Alternative exon 24 of myosin phosphatase regulatory subunit (Mypt1 E24) is a target as toggling between the two isoforms sets smooth muscle sensitivity to vasodilators such as nitric oxide (NO). The example relates to a gene based therapy to suppress splicing of Mypt1 E24 thereby switching MP enzyme to the NO-responsive isoform. Crispr/Cas9 constructs were effective at editing of Mypt1 E24 in vitro, however AAV9 was inefficient at targeting vascular smooth muscle in vivo. In contrast, an octo-guanidine conjugated anti-sense oligonucleotide targeting the 5′ splice site of Mypt1 E24 reduced percent splicing inclusion of Mypt1 E24 from 80 to 10% in mesenteric arteries. The maximal and half-maximal effects occurred at 12.5 and 6.25 mg/kg, respectively. The effect persisted for at least one month with no evidence of toxicity. This highly effective splice blocking anti-sense oligonucleotide is a novel therapy to reverse vascular dysfunction common to diseases such as hypertension and heart failure.

### Methods

### Animals

The animal protocols used in this study were reviewed and approved by the Institutional Animal Care and Use Committee (IACUC) at the University of Maryland and adhere to NIH guidelines. For tissue harvest mice were euthanized via CO₂ inhalation followed by cervical dislocation. Tissues were quickly dissected and either placed into RNAlater (Invitrogen) for later purification of RNA, frozen in liquid nitrogen and stored at −80° C. for protein analysis, or placed on ice cold PBS for imaging of fresh intact tissues.

### Crispr Cas9 In Vitro

Design and cloning of sgRNAs: Design of the sgRNAs was performed on the Benchling.com platform using the Design CRISPR Guides function. The target region on human PPP1R12A gene was set from 155881 bp to 156420 bp (gene sequence ENSG00000058272) allowing the screening of a 540 bp-long region flanking PPP1R12A exon 24 for potential sgRNA targets. Guides were subsequently evaluated for potential use in the mouse and rat by aligning the sequence of each sgRNA with the PPP1R12A murine sequences using MEGA 7.0.21 software. Guides with predicted efficiency and specificity scores exceeding 10% and 60%, respectively, alignments showing less than 8 mismatches between the sgRNA sequence and the human and murine PPP1R12A sequences and targeting a region immediately upstream a NNGRRT PAM sequence in all 3 species of interest, were selected for experimental in vitro evaluation.

sgRNA sequences were ordered from Integrated DNA Technologies with 5′-CACC and 5′AAAC overhangs to the sense and antisense oligos, respectively, with a G nucleotide added to the 5′ end of the sgRNA if one was not present to enhance transcription from the U6 promoter. sgRNAs were annealed and cloned into the px601 vector according to the ZhengLab protocol (available online http://www.genome-engineering.org/crispr/wp-content/uploads/2014/05/CRISPR-Reagent-Description-Rev20140509.pdf). px601-AAV-CMV::NLS-SaCas9-3xHA-bGHpA;U6::BsaI-sgRNA (px601) vector containing SaCas9 and a customizable U6-driven sgRNA scaffold (Pubmed25830891) was obtained from Addgene (Plasmid #61591). The sgRNA-px601 and control empty vector constructs were prepared by bacterial culture and purified using ChargeSwitch-Pro Plasmid Miniprep kit (ThermoFisher Scientific) by standard methods and according to manufacturer's protocol. Plasmids were sequenced using U6-Forward primer: 5′-GAG GGC CTA TTT CCC ATG ATT CC-3′ (SEQ ID NO:5) in order to confirm the presence of the sgRNA inserts in px601 vector.

Transfection experiments and analysis of editing of Mypt1 E24 HEK293T cells were seeded at a density of 10⁵ cells/well in 24-well plates. After 24 hours at approximately 80% density cells were transfected in duplicate with lug of plasmid using Lipofectamine™ 3000 Reagent (Life Technologies) according to manufacturer's protocol. Cells were harvested 72 hours later and genomic DNA purified using PureLink™ Genomic DNA Mini Kit (Invitrogen) per manufacturer's protocol.

AAV and injections AAV9: pAAV.CMV.HI.eGFP-Cre. WPRE.SV40 (from Addgene #105545) in which the CMV promoter and enhancer drives expression of EGFP and Cre recombinase. E24 f/f mice were injected IP with rAAV (2.67×10¹³ gc/kg) or empty vector at PND8-12 and assayed 2-3 weeks post-injection.

ASOs were Purchased from GeneTools LLC.

DNA Assays In order to investigate if genome editing occurred, locus PCR amplifying a 754pb-region flanking the exon 24 of PPP1R12A gene was performed using primers MYPT12AE24F 5′-ATGTTTAGGCATGCCGATGT-3′ (SEQ ID NO:6) (intronic region 155894 bp to 155913 bp on PPP1R12A ENSG00000058272) and MYPT12AE24R 5′-GCTTTGACTTTCTGGGAAGATG-3′ (SEQ ID NO:7) (intronic region 156626 bp to 156647 bp on PPP1R12A ENSG00000058272) under standard cycling conditions followed by resolution of PCR products by 2% agarose gel electrophoresis. This assay was complemented by the surveyor nuclease assay using the Surveyor® Mutation Detection Kit (Integrated DNA technologies) as per manufacturer's protocol. The Surveyor® Mutation Detection Kit uses an endonuclease that cleaves DNA with high specificity at sites of mismatches and other distortions, allowing the detection of known and unknown DNA mutations (PMID 15088388). Finally in some instances DNA was sequenced to identify Crsipr/Cas9 induced mutations.

### Results

### Efficient Crispr/Cas9 Editing of Mypt1 E24 In Vitro

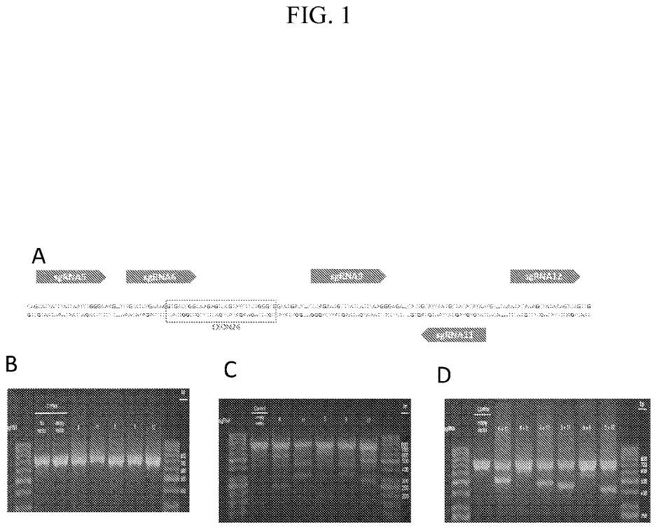

A total of 13 sgRNAs were identified by Benchling for potential use to target PPP1R12A E24 and intronic flanking sequence. Four sgRNAs were eliminated for low predicted efficiency scores (sgRNAs 1, 4, 8, 10). Five other sgRNAs (sgRNAs 2, 3, 7, 9, 13) were eliminated due to important mismatches between human and murine DNA sequences and/or lacking PAM in the murine sequences, based on alignment results (data not shown). Five sgRNAs were selected for further evaluation individually and in combination with other sgRNAs with the goal of total excision of E24 (FIG. 1). When sgRNA-px601 constructs were tested individually, the differences in size of genomic DNA induced by the edits were not resolvable under the conditions of agarose gel electrophoresis. The surveyor endonuclease assay yielded 2 bands by gel electrophoresis reflecting the formation of 2 products after cleavage of heteroduplexes generated by hybridization of control DNA and DNA edited by sgRNA-px601 constructs while only 1 product was observed in control DNA. Genomic edits which sizes were approximately 500 bp, 600 bp, 470 bp, 450pb, 570 bp and 420 bp, were observed for cells transfected with duets 6+11, 6+9, 6+12, 5+11, 5+9 and 5+12 of sgRNA-px601 constructs, respectively. Efficiency of editing ranged from 10% (sgRNAs 6+9 and 5+9) to 40% (sgRNAs 6+11 and 5+11). This represents an under-estimate of approximately ½ in the efficiency of editing since only ~50% of the cells in the culture were transfected as indicated by a fluorescent reporter (data not shown).

Because splicing of Mypt1 E24 is not present in cultured cells, including cultured SMCs, the effect of the Crispr/Cas9 gnomic editing on splicing of E24 could not be assessed in vitro. Thus, plasmids containing the most effective sgRNA (5,6,11) were used to generate recombinant AAV serotype 9. These AAV when injected into mice did not induce editing of Mypt1 E24 in the target SMCs in any tissue within the mouse (data not shown). Thus a series of experiments were performed to test recombinant AAV targeting of SMCs and the E24 target.

### Anti-Sense Oligonucleotides Efficiently Block Splicing of Mypt1 E24

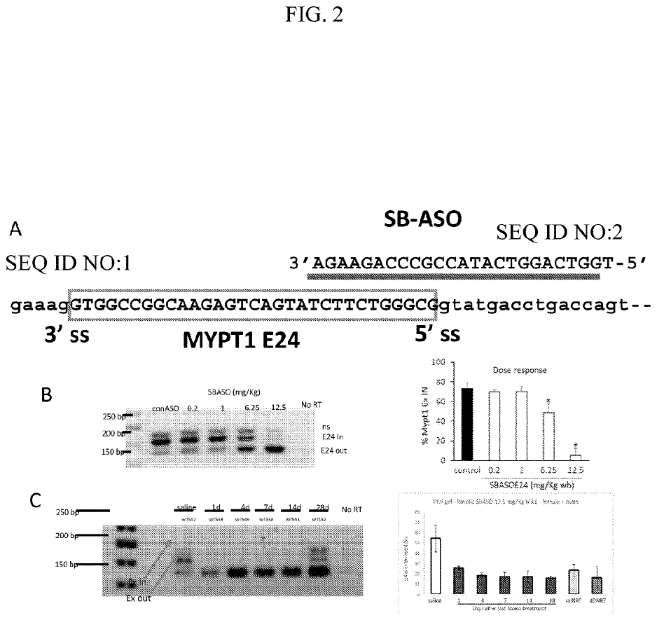

Splice blocking anti-sense oligonucleotides are an alternative strategy to viral transduction of Crispr-Cas9 for the purpose of shifting isoform expression for therapeutic gain. For this purpose we tested splice-blocking, i.e. RNAseH-independent, ASOs synthesized by GeneTools LLC (“morpholinos”). The morpholino was designed to hybridize with the 5′ splice site of Mypt1 E24. Preliminary studies showed that it was highly effective against its target when injected IP or IV into neonatal or mature mice at the standard dose of 12.5 mg/kg (data not shown). This was followed by dose-response and kinetic studies. SBASOE24 nearly completely suppressed splicing of Mypt1 E24 in the mesenteric arteries when injected at a dose of 12.5 mg/kg IP every other day for three doses and assayed on the seventh day (FIG. 2). As is also evident in the gel, there was a switch to the splice variant of Mypt1 lacking E24 with no significant change in the level of total Mypt1 (E24+ plus E24− variants). There was a good dose-response relationship, with EC₅₀ at 6.25 mg/kg and no effect at a dose of 1 mg/kg or lower (FIG. 2).

The effect of SBASOE24 on splicing of Mypt1 E24 was prolonged (FIG. 2). After three injections IP of 12.5 mg/kg over six days, the effect of SBASOE24 on splicing of Mypt1 E24 in mesenteric arteries persisted for 28 days (FIG. 2). longer timepoints have not been examined. In limited studies the effect of SBASOE24 on splicing of Mypt1 E24 was specific, as there was no effect on the splicing of the alternative exons of other SMC contractile mRNAs. The effect of SBASOE24 on Mypt1 E24 was not a non-specific effect of the ASO, as a control ASO had no effect on its splicing.

There was no evidence of toxicity of the SB-ASO at the dose used. After two weeks of injection at 12.5 mg/kg three times per week for two weeks, there was no increase in serum liver enzymes that would indicate liver injury, and no increase in serum creatinine that would indicate kidney dysfunction. There were also no increase in serum cytokines that would indicate immune activation, nor any indication of tissue damage nor inflammatory infiltrates on histological examination of the liver and kidney.

### Example 2. MyoNOsen, a Novel Oligonucleotide Therapy for Hypertension and its Sequela, Heart Failure (HFpEF)

Hypertension affects up to ½ the adult population and is the #1 treatable cause of early mortality through its sequelae of heart failure (HF), kidney failure, and stroke. Half of all patients are not optimally controlled with current therapies. The price of hypertension is staggering, totaling nearly $200 billion dollars in the US. To address these unmet needs, we are developing MyoNOsen, a long-lasting injectable oligonucleotide drug with a unique and powerful mechanism of action for treating patients with hypertension and “preserved Ejection Fraction” subtype of HF (HFpEF). MyoNOsen works by entering smooth muscle cells of arteries, where it binds with high efficiency to the pre-messenger RNA that encodes the Mypt1 subunit of myosin phosphatase, a key regulator of muscle contraction. MyoNOsen is designed to shift the balance between two natural variants of Mypt1, favoring the one that lacks Exon 24 (E24-). Unlike Mypt1/E24+, Mypt1/E24-is sensitive to vasodilators like Nitric Oxide (NO). By making this change, MyoNOsen allows the body's own pro-vasodilation molecules to be more effective at causing relaxation of arterial smooth muscle, which directly lowers blood pressure. Our preliminary studies in mice show MyoNOsen lowers Mypt1/E24+ and raises Mypt1/E24-without causing obvious side-effects. In these studies, the effects of one course of MyoNOsen lasted at least a month. Ultimately, we expect MyoNOsen to achieve the same durability as Leqvio® (inclisiran), a twice-yearly injectable oligonucleotide drug that was recently approved by the FDA to lower cholesterol. This example will determine its maximum tolerated dose in mice and look for pathological side-effects in major organs. This example will confirm MyoNOsen's effects on Mypt1 expression. Then, in two well-understood mouse models of circulatory disease, the example will address whether this drug candidate can reduce high blood pressure and treat HFpEF.

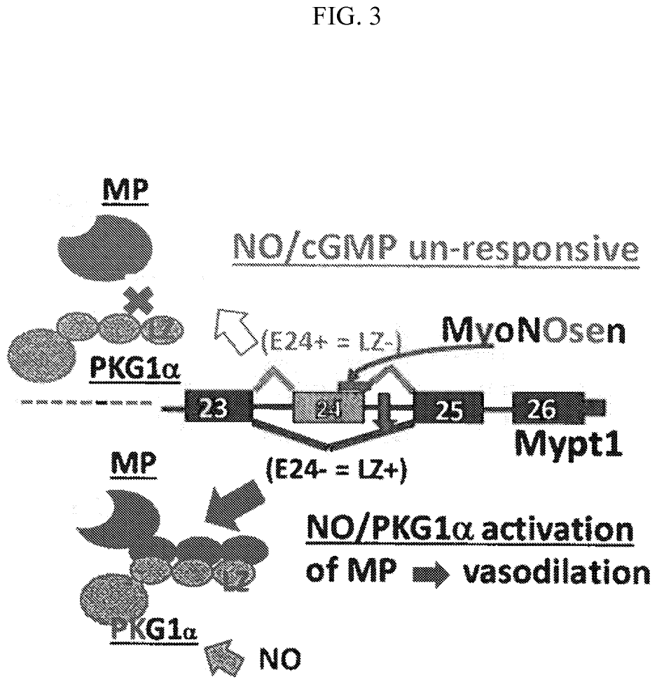

MyoNOsen is an ON whose sequence covers the 5′splice site of Exon 24 of the Mypt1 pre-mRNA. By binding there, it favors the formation of the naturally-occurring E24-minus (E24-) splice variant. This mRNA encodes the Mypt1 leucine zipper positive (LZ+) protein isoform, which includes a C-terminal leucine zipper (LZ) motif. (In the absence of MyoNOsen, the E24− isoform codes for as little as 20% of Mypt1 protein 2). Dimerization of the Mypt1/E24−/LZ+ with the LZ of cGMP-dependent protein kinase (PKG1α) enables Nitric Oxide (NO) signals to activate myosin phosphatase (MP; FIG. 3). Within the smooth muscle cells lining blood vessels, de-phosphorylation of myosin is the nexus of many pathways for regulating vascular tone. By shifting the MP isozyme pool in these cells, MyoNOsen will sensitize arteries to vasodilation signals, thereby lowering blood pressure (BP).

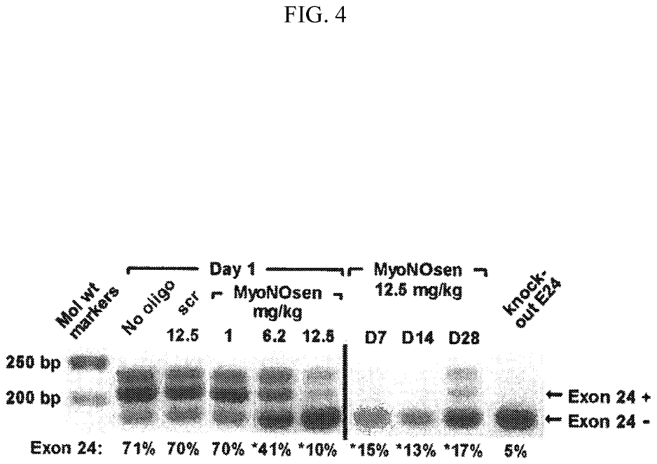

Our lab used genetically modified Cre/lox mice to demonstrate the effects of Mypt1 Exon 24 skipping on vascular tone. GeneTools LLC synthesizes 25-mer MyoNOsen as a phosphorodiamidate nucleotide conjugated to a string of 8 guanidines to improve cellular uptake7. C57B6/J mice were given 3 i.p. injections of MyoNOsen. Within a few days, a dose of 12.5 mg/kg had altered the splicing of Mypt1 pre-RNA in the small mesenteric arteries, while scrambled ON had no effect (FIG. 4). The oligo's splice-blocking effect lasted for 28 days.

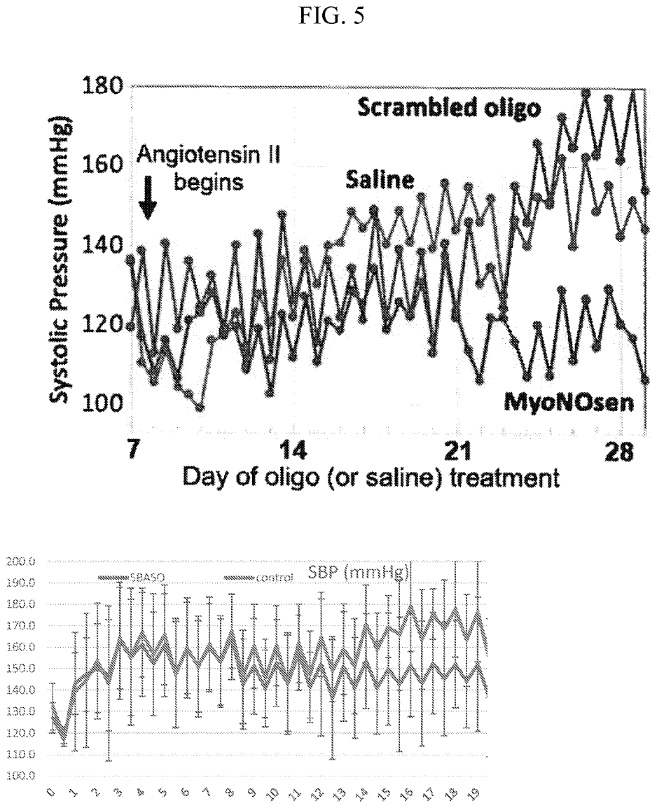

In a follow-on experiment, mouse BP was continuously recorded in ambulatory mice by implanted DSI PA-C10 telemetry devices, with HTN induced by infusion of angiotensin II (AngII). FIG. 5 presents average daytime and nighttime systolic BP. MyoNOsen suppressed the hypertensive response to chronic AngII infusion.

1) Synthesize MyoNOsen and control oligonucleotides. We will contract with GeneTools LLC to synthesize 2000 mmol (20 mg) of MyoNOsen, a 25mer Vivo-Morpholino oligonucleotide covering the 5′splice site of exon 24 of the Mypt1 pre-mRNA. An equal amount of control scrambled oligonucleotide (ON) will be synthesized and purchased.

2) Maximum Tolerated Dose of MyoNOsen. Mice will be injected with MyoNOsen or scrASO at maximal dose of 12.5 mg/kg qod (3×/week; n=5M,5F/group) throughout the 4 weeks of this experiment. Mice are observed and weighed weekly. After 4 weeks of treatment blood is collected, mice are euthanized and liver, kidney, heart, and aorta processed for H+E histology. Serum is assayed for kidney (Creatinine) and liver function tests (LFTs), red and white blood cell counts, and inflammatory markers (IL-1, IL-6, TNFa) using standard methods including plate-based assays and clinical labs. Potential off-targets were predicted by NCBI BLAST search (::: 13 consecutive nt, n=l 2) and will be assayed by PCR in the same manner as the target, Mypt1 E24.

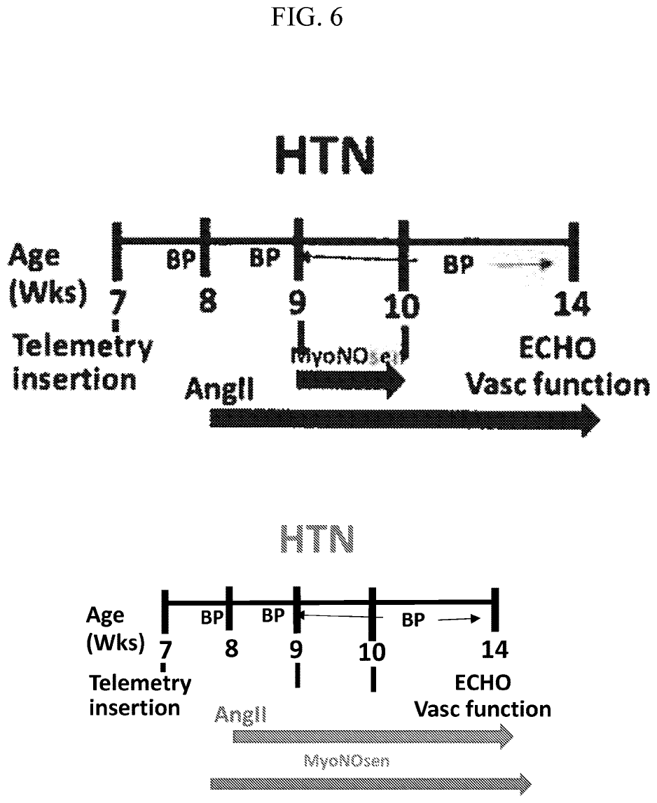

3) MyoNOsen as a novel therapy for HTN. Hypertension will be induced in wild type C57B6/J male mice aged 7-12 weeks by subcutaneous infusion of Angll at ~490 ng/kg/min. This is the chronic slow pressor model of HTN in which elevated BP develops after ~1 week and is maintained for the course of the infusion. The sequence of events is shown in FIG. 6: DSI PAC10 telemetry devices are surgically inserted into the carotid artery. Mice are allowed to recover; 1 week later baseline BPs are obtained followed by subcutaneous implantation of Alzet osmotic mini-pumps loaded with Angll for a calculated release rate of ~490 ng/kg/min. Mice are randomized to treatment with MyoNOsen or scrASO (12.5 mg/kg IP qod x 3, n=8 each). Ambulatory BP is continuously recorded via telemetry for the following 4 weeks. Just prior to euthanasia, echocardiograms (ECHO) are performed under anesthesia to assess cardiac function. Arteries and other smooth muscle tissues (gut, bladder) are recovered on necropsy to 1) assess effect of MyoNOsen on its target 2) assess vascular function by wire myography. BP is the primary endpoint and reported as SBP/MAP day and night-time averages over the entire course of the experiment. A preliminary power analysis indicates that. These experiments are approved under IACUC protocol #1021004 at UMB.

4) MyoNOsen as a novel therapy for HFpEF. HFpEF is essentially a complicated form of HTN in which advanced age, obesity and female sex are often components. This is modeled in mice as described (Withaar e al., Cardiovascular Research 2020). Wild type C57B6/J female mice aged 17.5 months have telemetry devices inserted and baseline BP recorded as above. Mice are placed on high fat diet: 60% of kcal from fat (Research Diets Inc. D12492)×8 weeks and another baseline BP obtained. They are then implanted with Alzet osmotic mini-pumps to release AngII at 800 ng/kg/min while maintained on HFD. 4 weeks later ECHO is performed to confirm HFpEF as evidenced by increased doppler derived diastolic filling pressures and increased LV thickness. Mice that meet criteria for HFpEF are randomized to MyoNOsen or control (scrASO) (12.5 mg/kg IP qod x 3) and continued on protocol with continuous telemetric monitoring of BP for 4 additional weeks. At that time heart failure is evaluated by 1) run test (Columbus Instruments) as described (Schiattarella G G et al., Nature, (2019), 568:351-6) 2) repeat ECHO to assess cardiac function 3) lung wet weights indicating pulmonary edema. Mice are then processed as in Task 3.

### Example 3. Targeting of a Splice Variant of the Regulatory Subunit of Myosin Phosphatase for Therapeutic Gain in Hypertension and Heart Failure

Myosin phosphatase (MP) is a serine/threonine protein phosphatase composed of catalytic (PPP1c), regulatory/targeting (Mypt1=PPP1R12a), small (M21=PPP1R12b) and inhibitory (CPl-17=PPP1R14a) subunits. MP is the primary regulator of smooth muscle force and a critical end target of nearly all signals that regulate vascular smooth muscle tone and thus blood flow and pressure. Nitric oxide (NO) signaling through the 2nd messenger cGMP and its kinase, cGMP-dependent protein kinase 1 (PRKG1α), binding to the regulatory subunit, activates MP, resulting in vasodilation, lowering of blood pressure and increased blood flow. Catecholamines, endothelin and other ligands for G-protein-coupled receptors (GPCRs), signaling through their 2nd messengers, and phosphorylation of inhibitory and/or regulatory subunits, inhibits MP, resulting in vasoconstriction. Phenotypically diverse and diseased smooth muscle tissues vary in their responses to these signaling pathways. Quite some time ago we hypothesized that this variability in response may be programmed into the MP, since it is the primary determinant of smooth muscle tone and an end-target/integrator of activating and inhibitory signals. We have focused on the 3′ 31 nt alternative exon24 (E24 of 26) of Mypt1. Skipping of E24 codes for a C-terminal leucine zipper motif (LZ) that is required for hetero-dimerization with PRKG1a and its activation of MP. Inclusion of 31 nt E24 changes the reading frame and introduces a premature termination codon, thereby encoding an entirely different C-terminal sequence (designated LZ-). This isoform renders the enzyme un-responsive to NO/cGMP mediated activation required for vasodilation. We have characterized the developmental and tissue-specific expression of these isoforms, their modulation in disease, and biochemical and physiological assays that support the working model. To test our working model in vivo, we generated mice with LoxP sequences flanking E24 and tamoxifen-inducible smooth muscle-specific Cre (smMHCCreER2). Treatment of homozygous Cre-Lox mice with tamoxifen (50 mg/kg IP qd×3) was highly effective: small mesenteric artery isoforms shifted from ~80% E24+/LZ− in wt to <10% E24+/LZ− in conditional knockout (cKO) mice. There were no other changes in MP subunit expression nor other non-specific effects. cKO mice had a suppressed hypertensive response to AngII delivered for 2 weeks by Alzet osmotic mini-pump at −500/ng/kg/min. As predicted mesenteric arteries from cKO mice were 10-100 fold more sensitive to NO/cGMP mediated vasorelaxation when studied ex vivo by wire myography.

We are now focused on translational approaches to shift the expression of these naturally occurring isoforms of Mypt1 to achieve NO/vasodilator sensitization as a novel approach to treat hypertension and heart failure. We have tested both CRISPR/Cas9 for permanent deletion of E24 and a splice blocking anti-sense oligonucleotide (SB-ASO) as a transient effect. While Crispr/Cas9 was efficient in vitro, inefficient viral (AAV) delivery to target smooth muscle cells (SMC) in vivo severely limited this approach. In contrast, an anti-sense oligonucleotide was highly effective in the suppression of E24 splicing in vivo when delivered by intra-peritoneal or intravenous injection of young and mature mice. Dose-response experiments indicated the efficacy at clinically relevant doses of 6.25-12.5 mg/kg. Pharmacodynamic experiments show a persistent suppression of Mypt1E24+/LZ-isoform for 1 month after loading with 12.5 mg/kg IP qod x 3. We conclude that targeting of Mypt1 E24, by causing a shift in naturally occurring MP isozymes, sensitizes vascular smooth muscle to NO/cGMP mediated vasorelaxation and could be a novel therapy for humans with hypertension and heart failure.

### Example 4. Development and Testing of MyoNOsen, 25Mer Vivo-Morpholino Oligonucleotide

MyoNOsen is a 25mer Vivo-Morpholino oligomer with a sequence designed to cover the 5′ splice site of Exon 24 of the Mypt1 pre-mRNA. By binding there, it favors the formation of the naturally-occurring E24-splice variant. This mRNA encodes the Mypt1 LZ+protein isoform, which includes a C-terminal leucine zipper (LZ) motif (in the absence of MyoNOsen, the E24-isoform makes up as little as 20% of Mypt1 protein). Heterodimerization of the Mypt1/E24-LZ with the LZ of cGMP-dependent protein kinase (PKG1a) enables Nitric Oxide (NO) signals to activate myosin phosphatase (FIG. 3). Within the smooth muscle cells that line arteries and arterioles, de-phosphorylation of myosin is the convergence point of many pathways for the regulation of vascular tone and thus blood pressure.

The causes of uncontrolled HTN are complex and include the need to take multiple medicines by mouth on a daily or more basis, and limited efficacy with significant side-effects of available small molecule drugs. In some embodiments, MyoNOsen would be a first-in-class oligonucleotide for the treatment of HTN/HFpEF with unique features: it is an injectable with prolonged duration of action (>1 month), uniquely targets the end-effector of a critical signaling pathway, and uniquely sensitizes blood vessels to beneficial endogenous vasodilator signals. MyoNOsen targets alternative exon 24 (E24) of the smooth muscle myosin phosphatase regulatory subunit (Mypt1=PPP1R12a). Myosin phosphatase (MP) by MyoNOsen, by steric blocking of splicing of E24, causes a shift to the naturally occurring Mypt1 E24 skipped mRNA coding for the C-terminal leucine zipper motif (LZ) that is required for NO/cGMP/activation of MP via binding of cGMP-dependent protein kinase (PKG1a). In small arteries that control vascular resistance and thus blood pressure/flow, this normally constitutes ~20-40% of the MP isozyme pool. By shifting the MP isozyme pool MyoNOsen will sensitize small arteries to upstream vasodilator signals thereby lowering blood pressure and augmenting blood flow.

MyoNOsen is synthesized and provided as a powder for re-constitution in water. Key features of this 25mer oligonucleotide (ON) include phosphorodiamidate nucleotide linkages to improve stability and octo-guanidine conjugation for improved cellular uptake1 https://www.gene-tools.com/). Injection of MyoNOsen at a standard dose (12.5 mg/kg IP (or IV, not shown) qod×3 caused near complete suppression of E24 splicing in mesenteric arteries (MA) of male and female mice (FIG. 4). There was a dose-response with ED₅₀ of ~6.25 mg/kg, a clinically relevant dose⁹. Injection of scrambled ON had no effect. The splice blocking effect of MyoNOsen persisted for 28 days after treatment (12.5 mg/kg IP qod×3) ended. We have not assayed longer time points. There was no evidence of non-specific effects on mRNA nor generalized toxicity (not shown). Thus, MyoNOsen is highly effective against its target in vivo with long lasting effect and no general toxicity.

DSI telemetry devices were surgically implanted into the carotid arteries for state-of-art but costly yet only reliable method for assessing BP in mice. There were 2 groups of n=5-6 mice each: 1) EXP: MyoNOsen 12.5 mg/kg IP 3x/wk 2) Control: scrambled ASO 12.5 mg/kg IP 3x/wk or Vehicle (saline) control IP 3x/wk. Mice were given AngiotensinII at 490 ng/kg/min by subcutaneous implantation of Alzet osmotic mini-pump. BP/HR were continuously recorded over 28 days in ambulatory mice and analyzed as average day-/night-time systolic (SBP), diastolic and mean blood pressure (FIG. 5). MyoNOsen suppressed the hypertensive response to chronic AngII infusion with separation of the groups beginning at 14 days and clear differences out to 20 days after initiation of AngII (FIG. 5).

This data is supported by experiments in which we used genetically modified Cre-lox mice to achieve the same effect of removal of E24 from Mypt1 (E24 conditional knock-out=E24cKO). E24cKO mice had suppressed hypertensive response in this same AngII model of HTN¹⁰. MAs from E24cKO mice have increased sensitivity to NO/cGMP mediated vasorelaxation as predicted by the model and likely accounting for the reduction in BP. In summary: experiments support E24 as a bona-fide novel target for treatment of HTN. The data shows that MyoNOsen is highly effective in vivo against its target, and that suppression of E24 lowers BP in HTN.

While the present teachings are described in conjunction with various embodiments, it is not intended that the present teachings be limited to such embodiments. On the contrary, the present teachings encompass various alternatives, modifications, and equivalents, as will be appreciated by those of skill in the art.

Throughout this disclosure, various publications, patents and published patent specifications are referenced by an identifying citation. The disclosures of these publications, patents and published patent specifications are hereby incorporated by reference into the present disclosure to more fully describe the state of the art to which this invention pertains.

1. An oligomer capable of binding to a target site of precursor messenger RNA (pre-mRNA) of myosin phosphatase target subunit (Mypt1) in cells, wherein binding of the oligomer to the target site of the pre-mRNA suppresses splicing of exon 24 of myosin phosphatase target subunit (Mypt1) in cells.

2. The oligomer of claim 1, wherein the target site of the pre-mRNA comprises a splice site of exon 24.

3. The oligomer of claim 1, wherein the oligomer binds to at least 20 contiguous nucleotides of the pre-mRNA.

4. The oligomer of claim 1, wherein the target site comprises at least 20 contiguous nucleotides of SEQ ID NOS: 1, 8 or 9.

5. The oligomer of claim 1, wherein the splice site of exon 24 is the 5′ splice site.

6. The oligomer of claim 1, wherein the oligomer comprises a nucleotide sequence that is complementary to the sequence of the target site.

7. The oligomer of claim 1, wherein the oligomer comprises a sequence that is complementary to the target site except at up to two base positions, wherein the oligomer binds to the target site and suppresses splicing of exon 24.

8. The oligomer of claim 1, wherein the oligomer is a phosphorodiamidate morpholino oligonucleotide (PMO) or a phosphorothioate-linked 2′-O-methyl oligonucleotide (2′OMePS).

9. (canceled)

10. The oligomer of claim 1, wherein the oligomer is between 12 and 40 bases in length.

11. (canceled)

12. The oligomer of claim 1, wherein the oligomer comprises a base sequence of SEQ ID NO:2 or SEQ ID NO:10.

13. The oligomer of claim 1, wherein the oligomer is conjugated to or complexed with a chemical moiety.

14. The oligomer of claim 12, wherein the oligomer is conjugated to octo-guanidine.

15. A pharmaceutical composition comprising an effective amount of the oligomer of claim 1.

16. A method of lowering blood pressure in a subject, comprising administering to the subject an effective amount of the pharmaceutical composition of claim 15, thereby lowering blood pressure in the subject.

17. A method treating hypertension in a subject, comprising administering to the subject an effective amount of the pharmaceutical composition of claim 15, thereby treating hypertension in the subject.

18. A method of treating heart failure in a subject, comprising administering to the subject an effective amount of the pharmaceutical composition of claim 15, thereby treating heart failure in the subject.

19. The method of claim 16, wherein the method further comprises administering to the subject an effective amount of one or more treatments for lowering blood pressure in the subject.

20. The method of claim 19, wherein the one or more treatments comprises administering an agent that lowers blood pressure.

21. The method of claim 20, wherein the agent is selected from the group consisting of a diuretic, beta-blocker, alpha-blocker, alpha/beta blocker, centrally acting sympatholytic, peripherally acting sympatholytic, calcium-channel blocker, dihydropyridine, direct vasodilator, activator of the nitric oxide pathway, angiotensin-converting enzyme (ACE) inhibitor, and combinations thereof.

22. The method of claim 16, wherein the method results in a decreased incidence or probability of heart disease in the subject,
wherein the method results in a decreased incidence or probability of a heart attack in the subject, and/or
wherein the method results in a decreased incidence or probability of a stroke in the subject.

23.-34. (canceled)
